(19) United States
(12) Patent Application Publication
(10) Pub. No.: US 2008/0038338 A1
(43) Pub. Date: Feb. 14, 2008

(54) Tetracycline Package Formulations

(76) Inventors: Alexander D. Smith, Cary, NC (US); Joe Cobb, Greenville, NC (US); John Bettis, Greenville, NC (US); William C. Stagner, Apex, NC (US); Brad Gold, Greenville, NC (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/763,860

(22) Filed: Jun. 15, 2007

Related U.S. Application Data
(60) Provisional application No. 60/813,949, filed on Jun. 15, 2006.

(51) Int. Cl. A61K 9/24; A61K 31/165; A61P 29/00

(52) U.S. Cl. 424/464; 514/152

(57) ABSTRACT

The invention provides a rapidly disintegrating and dissolving multilayer tablet comprising at least a tetracycline in a first layer, a buffer in a second layer, and optionally, an inert layer separating the first and second layers. The multilayer tablets of the invention are useful for treating or preventing mucositis, when administered topically to the oral cavity.

This application claims priority from U.S. Provisional Application No. 60/813949, filed Jun. 15, 2006, the disclosure of which is incorporated by reference in its entirety.

### BACKGROUND OF THE INVENTION

1. Field Of The Invention

The invention relates to rapidly disintegrating and dissolving solid dosage. More specifically, it relates to such dosage forms containing a tetracycline, buffer, and an optional inert layer separating the tetracycline and the buffer. These dosage forms are useful for treating or preventing mucositis, when administered topically to the oral cavity.

2. Description of the Related Art

Tetracycline is a broad-spectrum antibiotic from the certain streptomyces species. Tetracycline is typically used to treat bacterial infections, such as infections of the skin, respiratory tract, genital and urinary systems, and stomach. Tetracycline is also used to treat Lyme disease. Tetracycline works by preventing the growth and spread of bacteria.

Tetracycline antibiotics degrade rapidly to form epitetracycline, anhydrotetracycline, epianhydrotetracycline, and other degradation products. Once degraded, tetracycline has no therapeutic value, since the degradation products have no therapeutically useful activity. Tetracycline degradation begins as soon as it is in solution, and generates epimeclocycline as a primary degradant. More epimer is formed at higher temperatures and lower pH. Oxidation and other side reactions cause further degradation. Thus, tetracycline products have a very limited existence in aqueous environments. Consequently, tetracycline cannot be stored in solution for extended periods of time.

Therefore, there is a need for tetracycline formulations that remain therapeutically effective during long term storage.

### SUMMARY OF THE INVENTION

The invention provides a pharmaceutical composition that is generally a multilayer tablet, the tablet comprising (1) a layer of a tetracycline, preferably a poorly absorbable tetracycline such as meclocycline, more preferably meclocycline sulfosalicylate, preferably with excipients, (2) an optional intervening layer that serves as a barrier, and (3) a layer containing a buffer or base, and preferably, excipients. The intervening barrier layer, if present, can be a coating that surrounds a core layer of buffer or meclocycline sulfosalicylate. Alternatively, the intervening barrier layer can be situated between a layer of buffer and a layer of meclocycline sulfosalicylate in a sandwich type format. The intervening barrier layer prevents contact between the buffer and the meclocycline sulfosalicylate. In certain aspects, the tablet rapidly disintegrates upon contact with an aqueous medium.

The invention also provides methods for using a multilayer tablet of the invention for treating or preventing mucositis, comprising orally administering the multilayer tablet directly to a patient, or comprising mixing the multilayer tablet with an aqueous medium and contacting the resulting solution with a patient's oral cavity.

The invention further provides an aqueous formulation comprising (a) a solution phase comprising water, tetracycline and a buffer, and (b) a solid phase present or suspended in the solution phase, the solid phase comprising water insoluble material.

Specific preferred embodiments of the invention will become evident from the following more detailed description of certain preferred embodiments and the claims.

### DETAILED DESCRIPTION OF THE INVENTION

In one aspect, the invention provides a pharmaceutical composition in the form of a multilayer tablet comprising
(a) a first region comprising Formulation (A), wherein Formulation (A) comprises a therapeutically effective amount of a tetracycline, or a pharmaceutically acceptable salt thereof, in an amount of about 5% to about 40% by weight of Formulation (A), and a first carrier material comprising at least one pharmaceutically acceptable binder, carrier, adjuvant, excipient, diluent, disintegrant, lubricant or glidant; and
(b) a second region comprising with Formulation (B), wherein Formulation (B) comprises, a buffer and a second carrier material comprising at least one pharmaceutically acceptable binder, carrier, adjuvant, excipient, diluent, disintegrant, lubricant, or glidant, wherein the tablet rapidly disintegrates in an aqueous medium.

Preferred buffers include for use in Formulation (B) include tris(hydroxymethyl)aminomethane (tromethamine); monobasic phosphate salts such as monobasic sodium phosphate and monobasic potassium phosphate; dibasic phosphate salts such as dibasic sodium phosphate dibasic potassium phosphate, and dibasic sodium/potassium phosphate; tribasic phosphate salts, such as sodium phosphate tribasic, potassium phosphate tribasic, and tribasic sodium/potassium phosphate; sodium pyrophosphate; lysine; or a combination of at least two of the above.

Preferred second carrier material comprises a filler/binder/disintegrant, such as a cellulose derivative, e.g., hydroxymethylcellulose or microcrystalline cellulose; lactose; preferably lactose DT; pregelatinized starch; or corn starch; etc. A particularly preferred cellulose derivative is microcrystalline cellulose.

The second carrier material typically also comprises a disintegrant, such as croscarmellose sodium, microcrystalline cellulose, crospovidone, sodium starch glycolate, or combinations thereof. Croscarmellose sodium is preferred.

The second carrier material further typically comprises a lubricant, such as magnesium stearate, stearic acid, talc, or combinations thereof. Magnesium stearate is preferred.

In preferred aspects of the invention, the tetracycline is meclocycline or a salt thereof, preferably meclocycline sulfosalicylate.

The first carrier material generally comprises a filler/binder/disintegrant, such as a cellulose derivative, e.g., hydroxymethylcellulose or microcrystalline cellulose; lactose, preferably lactose DT; pregelatinized starch; corn starch; or combinations thereof.

The first carrier material further typically comprises a disintegrant, such as croscarmellose sodium, microcrystalline cellulose, crospovidone, sodium starch glycolate, or combinations thereof. Croscarmellose sodium is preferred.

The first carrier material generally also comprises a lubricant, such as magnesium stearate, stearic acid, talc, or combinations thereof. Magnesium Stearate is preferred.

In yet still another aspect, the tablet has a hardness of greater than 5 kp (preferably grater than 6 kp, more preferably grater than 7 kp), friability less than 0.5% (preferably less than 0.4%, more preferably less than 0.3%, still more preferably less than 0.2%).

Preferred tablets of the invention have a disintegration time in aqueous media, typically tap water, of less than about 30, more preferably about 15, and most preferably about 10 seconds.

In still another aspect, the first carrier material comprises a filler/binder/disintegrant, such as microcrystalline cellulose; a disintegrant, such as croscarmellose sodium; a lubricant, such as magnesium stearate, and a filler, such as lactose anhydrous high velocity.

In yet another aspect, the second carrier material is comprises a filler/binder/disintegrant, such as microcrystalline cellulose; a disintegrant such as croscarmellose sodium; and a lubricant, such as magnesium stearate; wherein the microcrystalline cellulose is silicified microcrystalline cellulose.

It has been surprisingly discovered that a tetracycline, preferably meclocycline, more preferably meclocycline sulfosalicylate, may be stably incorporated into a bilayer tablet where the tetracycline is contained in a tablet region that is in direct contact with a tablet region containing buffer.

Thus, in a preferred aspect of the invention, the composition is in the form of a bilayer tablet, wherein

the first carrier material comprises about 150-350 mg of microcrystalline cellulose, about 20-60 mg of croscarmellose sodium, about 0.1-5 mg of magnesium stearate, and about 20-60 mg of lactose anhydrous high velocity; and

the second carrier material comprises about 100-400 mg of microcrystalline cellulose, about 20-60 mg of croscarmellose sodium, and about 0.1-5 mg of magnesium stearate, wherein the microcrystalline cellulose is silicified microcrystalline cellulose.

In a preferred aspect, the buffer of Formulation (B) is sodium phosphate tribasic.

In still another aspect, the weight of the first region is equal to or greater than the weight of the second region.

In another preferred aspect, the first carrier material comprises about 200-300 mg of microcrystalline cellulose, about 30-45 mg of croscarmellose sodium, about 0.8-2.5 mg of magnesium stearate, and about 30-45 mg of lactose anhydrous high velocity;

the second carrier material comprises about 125-225 mg of microcrystalline cellulose, about 30-50 mg of croscarmellose sodium, and about 0.8-2.5 mg of magnesium stearate, wherein the microcrystalline cellulose is silicified microcrystalline cellulose;

the weight of the sodium phosphate tribasic is about 50-100 mg; and

the weight of the meclocycline is about 25-75 mg.

In another aspect, at least one of the regions contains at least one disintegrant.

The invention additionally provides a formulation of that

1) disintegrates within about 8 to 12 seconds of being added to an aqueous medium, preferably water, more preferably tap water, and

2) where about 90% of the tetracycline and buffer dissolves within about 30 seconds—when the aqueous medium is mixed. Mixing includes shaking, stirring, agitating, and/or swirling the solution, either by hand, or by using a mechanical device.

The invention further provides a package comprising instructions describing how to use the dosage forms of the invention in the treatment and/or prevention of oral mucositis and at least one multilayer tablet, as described herein. The instructions detail at least two of the following: how much aqueous medium to use, how many of each dosage form to place in the aqueous medium, how long to wait after placing the dosage form in the medium, how to mix the dosage form into the aqueous medium and how to use the resulting mixture.

The invention further provides a method of treating oral mucositis (OM), comprising administering the composition contained in the inventive tablet as an aqueous mixture to a patient in need of such treatment.

In a preferred aspect, the tablet is dissolved in water and then administered to the oral cavity of the patient.

In another aspect, the water containing the dissolved composition has a pH of about 6-10, preferably about 7-9 (or more preferably about 8-9).

The buffer helps maintain the pH of the aqueous medium and helps maximize the solubility and stability of the tetracycline (such as meclocycline sulfosalicylate) in the aqueous medium.

In a preferred aspect, the tablets are used to prepare an aqueous mouth rinse composition which is immediately, i.e., within about 5 minutes of preparation, used to rinse the oral cavity. More preferably, the composition is used to rinse the oral cavity within 3 minutes of adding the composition to the water. Still, more preferably, the composition is used to rinse the oral cavity within 1 minute of adding the composition to the water. To prepare the aqueous mouth rinse, the tablets are added to a predetermined amount, e.g., 5 ml, 10 ml, 15 ml, 20 ml, or 25 ml, of water, typically tap water, after which the water/tablet mixture may be mixed by stirring or shaking to disintegrate and dissolve the tablet components. In preferred aspects of the invention, the tetracycline (or salt thereof) and the buffer will dissolve in the water, while other components, in particular the disintegrants will be insoluble. In addition to the tetracycline and buffer dissolving, in preferred aspects, the lactose will also dissolve in the water.

In another aspect, the invention provides a composition the form of a trilayer tablet, comprising at least,

(a) a first region comprising Formulation (A), wherein Formulation (A) comprises, a therapeutically effective amount of a tetracycline, or a pharmaceutically acceptable salt thereof, in an amount of about 5% to about 40% by weight of Formulation (A), and a first carrier material comprising at least one pharmaceutically acceptable binder, carrier, adjuvant, excipient, diluent, disintegrant, lubricant, or glidant;

(b) a second region comprising with Formulation (B), wherein Formulation (B) comprises, a buffer and a second carrier material comprising at least one pharmaceutically acceptable binder, carrier, adjuvant, excipient, diluent, disintegrant, lubricant, or glidant, wherein the tablet rapidly disintegrates in an aqueous medium; and

(c) a third region, located between the first and second regions, where the third region comprises at least one pharmaceutically acceptable binder, carrier, adjuvant, excipient, diluent, disintegrant, lubricant, or glidant.

In one aspect, the third region is essentially inert.

In certain embodiments, the invention provides multilayer tablets that are designed for constitution as solutions by dissolution or suspension in a liquid vehicle, preferably water.

In another aspect, the invention provides an aqueous formulation comprising (a) a solution phase comprising a tetracycline (such as meclocycline or meclocycline sulfosalicylate) and a buffer dissolved in water, and (b) a solid phase present or suspended within the solution phase. The solid phase comprises water insoluble material, and the water insoluble material, typically at least partially in particulate or granular form, comprises tablet binder, carrier, adjuvant, excipient, diluent, disintegrant, glidant, lubricant, or a combination thereof, i.e., material produced by disintegration of the tablet but that is not soluble in water.

In a preferred aspect, the invention provides an aqueous formulation having a volume of from about 5-30 ml, more preferably from about 8-25 ml, and comprising (a) a solution phase comprising water, about 0.07-2% (w/w) of a tetracycline and about 0.1-4% (w/w) of a buffer, and (b) a solid phase present or suspended in the solution phase (both weight percentages being on the basis of the weight of the solution phase and not the solution phase plus the solid material), the solid phase comprising water insoluble material. Preferably the volume of the formulation is from about 10-20 ml, and most preferably about 15 ml. In preferred embodiments of this aspect the weight percentage of tetracycline in the solution phase is from about 0.1 to about 1.0. In other preferred embodiments of this aspect, the weight percentage of tetracycline in the solution phase is from about 0.15 to about 0.5. In other preferred embodiments of this aspect, the weight percentage of tetracycline in the solution phase is from about 0.22 to about 0.47. Preferably the water insoluble material is tablet binder, carrier, adjuvant, excipient, diluent, disintegrant, glidant, lubricant or a combination thereof. In one embodiment, the buffer in the solution phase of the aqueous formulation is tris(hydroxymethyl)aminomethane (tromethamine); a monobasic phosphate salt, such as monobasic sodium phosphate or monobasic potassium phosphate; dibasic phosphate salts, such as dibasic sodium phosphate, dibasic potassium phosphate, or dibasic sodium/potassium phosphate; tribasic phosphate salts, such as sodium phosphate tribasic, potassium phosphate tribasic, or tribasic sodium/potassium phosphate; sodium pyrophosphate; lysine; or a combination thereof.

In another embodiment, the water insoluble material comprises a filler/binder/disintegrant, such as a cellulose derivative, e.g., hydroxymethylcellulose or microcrystalline cellulose; etc. A particularly preferred cellulose derivative is microcrystalline cellulose.

In still another embodiment, the water insoluble material comprises a disintegrant, such as croscarmellose sodium, microcrystalline cellulose, crospovidone, sodium starch glycolate, or combinations thereof. Croscarmellose sodium is preferred.

In still another embodiment, the water insoluble material comprises a lubricant, such as magnesium stearate, stearic acid, talc, or combinations thereof. Magnesium Stearate is preferred.

In still another embodiment, the water insoluble material comprises microcrystalline cellulose (such as silicified microcrystalline cellulose), croscarmellose sodium, and magnesium stearate.

In one embodiment, the formulation further comprises lactose, which is dissolved in the water.

The aqueous formulation is used shortly after it is prepared, and preferably within about 5 minutes of its preparation.

The aqueous formulation may further comprise one or more flavoring agents, coloring agents, or combinations thereof.

To make the aqueous formulation, the tablet, such as the bilayer tablet, is added to about 5-30 ml of water (preferably about 9-20 ml, more preferably 15 mL), and then shaken for approximately 30 seconds. The tablet will disintegrate within about 5-20 seconds (preferably 6-15 seconds, more preferably about 8-12 seconds, and still more preferably about 10 seconds). Preferably, at least about 90% of the tetracycline, the buffer, and the lactose (if present) will dissolve within the approximately 30 seconds, after which the water insoluble material will still be visibly present. The water insoluble material may comprise particulates; therefore, the resulting aqueous formulation may appear cloudy.

If desired, the aqueous formulation may be filtered before use.

After making the aqueous formulation, the patient rinses her mouth with the solution for approximately 30 seconds. The patient may also use the solution as a gargle, in order to treat the back of the oral cavity, i.e., the upper region of the throat.

Preferably, the patient will not rinse her mouth for at least about 30 minutes after dosing. Additionally, it is preferable that the patient not eat or drink anything for approximately 30 before and approximately 30 minutes after rinsing with the aqueous formulation.

As used herein, “water” refers to distilled water, deionized water, bottled water, tap water, and water having salts, minerals, etc., dissolved in it.

Any configuration can be used for a multilayer tablet of the invention. For example, a multilayer tablet can have any geometric shape, such as a bi-convex. This can comprise an inner core that includes as a pharmaceutically active component meclocycline sulfosalicylate. Alternatively, the inner core can comprise a buffer, such as tromethamine. The inner core can be formulated by compressing the meclocycline sulfosalicylate or buffer in any suitable tabletting equipment using standard compression tabletting techniques.

In certain embodiments, a multilayer tablet of the invention also comprises an intervening barrier layer that separates meclocycline sulfosalicylate from buffer. An intervening layer may be comprised of common tabletting excipients such as microcrystalline cellulose, lactose, povidone, silicon dioxide, corn starch or pregelatinized starch, disintegrants like sodium starch glycolate, croscarmellose sodium, or crospovidone or any other number of commonly employed tabletting ingredients The barrier layer can surround the core or can be sandwiched between the meclocycline sulfosalicylate and buffer layers. The intervening barrier layer can be any suitable material that prevents interaction of the meclocycline sulfosalicylate and buffer. A suitable intervening barrier layer substantially prevents contact of meclocycline sulfosalicylate with the buffer/base. Preferably the intervening barrier layer can easily dissolve in an aqueous medium. In addition, the intervening barrier layer is preferably inert, so that it does not interact with either meclocycline sulfosalicylate or buffer.

Where the multilayer tablet comprises a core, the intervening barrier layer can be coated onto the inner core using standard coating techniques. For example, aqueous or solvent coating techniques can be used to apply the coating to the inner core.

In certain embodiments, an outer layer can surround the intervening barrier layer and inner core. Where the inner core is meclocycline sulfosalicylate, the outer layer comprises buffer. Where the inner core is buffer, the outer layer comprises meclocycline sulfosalicylate. The outer layer can be applied by compression coating or solvent coating techniques such as are well known in the tabletting art.

In one embodiment, the multilayer tablet forms a solution or suspension of meclocycline sulfosalicylate upon contact with an aqueous medium. The multilayer tablet includes meclocycline sulfosalicylate and a buffer that disintegrate in the aqueous medium to form a solution with a pH greater than about 5 and less than about 8. In one embodiment, the aqueous medium is saliva. In another embodiment, the aqueous medium is water in a volume of, for example, about 10-15 ml, in which the multilayer tablet rapidly disintegrates to form a mouth rinse in situ.

As used herein, “rapidly” generally means that the tablet dissolves or disintegrates within a short time, for example about two minutes, more preferably about 1 minute, and still more preferably about 30 seconds.

As used herein, “tetracycline” means tetracycline analogues and derivatives include the following: oxytetracycline; chlortetracycline; demeclocycline; doxycycline; minocycline; rolitetracycline; lymecycline; sancycline; tetracycline; methacycline; apicycline; clomocycline; guamecycline; meglucycline; mepyclcline; penimepicycline; pipacycline; etocycline, penimocycline, and meclocycline. Preferred tetracyclines include meclocycline.

The term “pharmaceutically acceptable salt” refers to those salts of tetracyclines that are not substantially toxic at the dosage administered to achieve the desired effect and do not independently possess significant pharmacological activity. The salts included within the scope of this term are pharmaceutically acceptable acid addition salts of a suitable inorganic or organic acid. Suitable inorganic acids are, for example hydrochloric, hydrobromic, sulfuric and phosphoric acids. Suitable organic acids include carboxylic acids, such as acetic, propionic, glycolic, lactic, pyruvic, malonic, succinic, fumaric, malic, tartaric, citric, cyclamic, ascorbic, maleic, hydroxymaleic, dihydroxymaleic, benzoic, phenylacetic, 4-aminobenzoic, 4-hydroxybenzoic, anthranillic, cinnamic, salicylic, 4-aminosalicyclic, 2-phenoxybenzoic, 2-acetoxybenzoic and mandelic acid, sulfonic acids, such as methanesulfonic, sulfosalicylate, ethanesulfonic and β-hydroxyethanesulfonic acid. Sulfosalicylate is a preferred salt. In addition, pharmaceutically acceptable salts include those salts of tetracyclines formed with inorganic and organic bases, such as those of alkali metals, for example sodium, potassium and lithium, alkaline earth metals, for example calcium and magnesium, light metals of group IIIA, for example aluminum, organic amines, for example primary, secondary or tertiary amines, such as cyclohexylamine, ethylamine, pyridine, methylaminoethanol and piperazine. The salts are prepared by conventional means by one of ordinary skill in the art as, for example, by treating a tetracycline with an appropriate acid or base. Such salts can exist in either a hydrated or substantially anhydrous form.

Active agents other than tetracycline can also be used in dosage forms of the invention to aid in the treatment or prevention of mucositis. These agents can be inflammatory cytokine inhibitors, and/or mast cell inhibitors and/or NO inhibitors that reduce and inhibit mucositis.

Agents that inhibit the function of the mast cells or the action of the mediators released by mast cells can be used to treat and prevent mucositis. Mast cell inhibitors are chemical or biological agents that suppress or inhibit the function of mast cells, or the mediators released by mast cells. For example, mast cell inhibitors can inhibit degranulation, thereby preventing the release of mediators into the extracellular space.

Examples of mast cell degranulation inhibitors include picetannol, benzamidines, tenidap, tiacrilast, disodium cromoglycate, lodoxamide ethyl, and lodoxamide tromethamine. Other agents that inhibit mediator release include staurosporine and CGP 41251. Examples of mast cell mediator inhibitors include agents that block the release or secretion of histamine, such as FK-506 and quercetin; antihistamines such as diphenhydramine; and theophylline. Other mast cell inhibitors include serine protease inhibitors, such as alpha-l-protease inhibitor; metalloprotease inhibitors; lisofylline; benzamidine; amiloride; and bis-amidines such as pentamidine and bis(5-amidino-2-benzimidazolyl)methane.

Inflammatory cytokine inhibitors are chemical or biological agents that suppress or inhibit inflammatory cytokines. Such inhibitors include pyridinyl imidazoles, bicyclic imidazoles, oxpentifylline, thalidomide and gabexate mesilate.

Anti-inflammatory agents can be used in combination with inflammatory cytokine and/or mast cell inhibitors to treat and prevent mucositis. Examples of anti-inflammatory agents that can be used include the non-steroidal anti-inflammatory drugs (NSAIDs) flurbiprofen, ibuprofen, ketoprofen, sulindac, and diclofenac. When NSAIDs are administered, anti-ulcer agents such as ebrotidine can be administered, e.g., to help protect against gastric mucosal damage. Other anti-inflammatory agents that can be used include misoprostil; methylxanthine derivatives, such as caffeine, lisofylline, or pentoxyfylline; benzydamine; naprosin; mediprin; and aspirin.

Another important class of anti-inflammatory agents includes cyclooxygenase (COX) inhibitors, particularly COX-2 inhibitors. COX-2 inhibitors that can be used include celecoxib, nimesulide, meloxicam, piroxicam, flosulide, etodolac, nabumetone, and 1-[(4methylsulfonyl)phenyl]-3-trifluoromethyl-5-[(4-fluoro)phenyl]pyrazole. Other useful anti-inflammatory agents include dual cyclooxygenase/lipoxygenase inhibitors, such as 2-acetylthiophene-2-thiazolylhydraregion, and leukotriene formation inhibitors, such as piriprost.

MMP inhibitors include both the antibacterial tetracyclines such as tetracycline HCl, minocycline and doxyocycline, as well as non-antibacterial tetracyclines.

Nitric oxide (NO) inhibitors can be any type. Preferable NO inhibitors can be aminoguanidine, guanidine or a mixture thereof.

The administration of anti-microbial agents in combination with the agents described above can result in an even more effective method for treating and preventing mucositis. Examples of antimicrobial agents that can be used include agents with activity against gram positive and gram negative organisms. Specific drugs include tetracycline HCl, amoxicillin, gentamicin, and chlorhexidine.

Other agents that may be used to treat or prevent mucositis in combination with tetracyclines include the nuclear transcription factor kappa-B (NF-B) activation inhibitors capsaicin and resiniferatoxin.

Other medicinal agents may be added to dosage forms of the invention for purposes of alleviating other undesirable conditions in the mouth. Such agents may include, for example, local anesthetics, antibacterial agents, and emollients, as well as anti-fungal agents.

The rapidly disintegrating multilayer tablet contains preferably 0.1-100.0 mg, more preferably 1 to 60 mg, and most preferably about 20-80, or 25-75, or 30-60 mg of meclocycline (as the sulfosalicylate salt). The rapidly disintegrating multilayer tablet also contains preferably 0.1-100.0 mg, more preferably 50 to 100 mg, and most preferably about 60-90 mg, of TRIS (tromethamine) buffer. The multilayer tablet may be added to a liquid vehicle to produce a mouth rinse. The mouth rinse is preferably prepared by the patient immediately prior to administration.

A mouth rinse composition can be administered to the oral cavity, held and swished around in the mouth, and then swallowed or spit out. The liquid vehicle is preferably water. Other components may be present in the vehicle as described, for example, in U.S. Pat. No. 6,683,067, which is incorporated by reference. Still other components may be present in the vehicle as described below.

In order to improve the patient acceptability, appropriate coloring and/or flavoring material can be added to a liquid vehicle before or after contact with the dosage forms of a package of the invention. Alternatively, the coloring and/or flavoring material can be added to the tablet. Any pharmaceutically acceptable coloring or flavoring material may be used. Flavorings used in the mouth rinse art such as peppermint, citrus flavorings, berry flavorings, custard, vanilla, cinnamon, and sweeteners, either natural or artificial, may be used. Flavorings that are known to increase salivary electrolyte concentrations may be added to increase the magnitude of the viscosity change.

In certain embodiments, a multilayer tablet of the invention comprises a buffer layer that dissolves in aqueous solutions to provide an optimal pH to permit solubility of the tetracycline and maintain the stability of the tetracycline (such as meclocycline sulfosalicylate). Procedures for choosing the optimum pH and buffering agents are well known. In a particular embodiment, the buffer in a multilayer tablet of the invention is tromethamine. Other factors that affect stability in solution are also well known. For example, antioxidants may be added to the multilayer tablet to reduce the rate of degradation due to oxidation.

Re-constitution of meclocycline sulfosalicylate via addition of a multilayer tablet of the invention to an aqueous based liquid can be accomplished, for example, by a patient or by a pharmacist prior to administration to use.

Once the oral rinse is prepared, it is normally dosed immediately or used with 5 minutes.

Unless otherwise required by context, singular terms as used herein shall include pluralities and plural terms as used herein shall include the singular.

The Examples that follow are merely illustrative of specific embodiments of the invention, and are not to be taken as limiting the invention, which is defined by the appended claims.

Methods of Preparing the Compositions

As used herein, a multilayer tablet is a tablet which is made up of two or more distinct layers or discrete regions of solid components or granulation compressed together with the individual layers lying one on top of another. Multilayer, preferably, bilayer tablets have the appearance of a sandwich because the edges of each layer or region is exposed. Such bilayer tablets are generally prepared by compressing a solid component mixture or granulation, e.g., Formulation A or Formulation B, onto a previously compressed component mixture. The operation may be repeated to produce tablets of more than two layers.

In a preferred embodiment of the present invention, the bilayer tablet consists of two layers wherein one layer is made from Formulation (A) and the other layer is made from Formulation (B). The first layer is compressed to the desired size using standard tooling. If present, the barrier layer is then compressed into the first layer. The second layer is then compressed into 1) the first layer or 2) the barrier layer, if present, at the desired target weight. The tablet may then be coated, if desired.

Presses that are useful in preparing trilayer tablets include HATA Models AP55-LSU-3L; AP45-LSU-3L; and AP71-LSU-3L.

Presses that are useful in preparing bilayer tablets include the Piccola bilayer press.

### EXAMPLES

### Example 1

A composition where the tetracycline (Meclocycline Sulfosalicylate) and the buffer (sodium phosphate tribasic) are intimately mixed.

| | Weight |
|---|---|
| INGREDIENT | Percent (%) |
| Meclocycline Sulfosalicylate | 9.36 |
| Sodium Phosphate Tribasic | 14.00 |
| Silicified Microcrystalline | 68.64 |
| Cellulose | |
| (PROSOLV SMSS 90, commercially | |
| available from JRS Pharma LP, | |
| Patterson, NY)) | |
| Croscarmellose Sodium, NF | 7.5 |
| (Ac-Di-Sol SD-711) | |
| Magnesium Stearate, NF | 0.50 |
| Total | 100 |

### Example 2

A bilayer tablet where the tetracycline (Meclocycline Sulfosalicylate) is in layer 1 and the buffer (sodium phosphate tribasic) is in layer 2.

| | Amount | Amount |
|---|---|---|
| | per | per |
| | Tablet | Tablet |
| | (mg) | (mg) |
| INGREDIENT | Layer 1 | Layer 2 |
| Meclocycline Sulfosalicylate | 46.8 | — |
| Sodium Phosphate Tribasic | — | 70.0 |
| Microcrystalline Cellulose PH200 | 251.3 | — |
| Silicified Microcrystalline | — | 183.5 |
| Cellulose | | |
| (PROSOLV SMSS 90, commercially | | |
| available from JRS Pharma LP, | | |
| Patterson, NY)) | | |
| Croscarmellose Sodium, NF | 37.5 | 45.0 |
| (Ac-Di-Sol SD-711) | | |
| Magnesium Stearate, NF | 1.9 | 1.5 |
| Lactose Anhydrous High Velocity | 37.5 | — |
| (DT) | | |
| Layer Weight in mg | 375 | 300 |

— = not present

### Example 3

A bilayer tablet where the tetracycline (Meclocycline Sulfosalicylate) is in layer 1 and the buffer (sodium phosphate tribasic) is in layer 2.

| | Amount | Amount |
|---|---|---|
| | per | per |
| | Tablet | Tablet |
| | (mg) | (mg) |
| INGREDIENT | Layer 1 | Layer 2 |
| Meclocycline Sulfosalicylate | 46.8 | — |
| Sodium Phosphate Tribasic | — | 70.0 |
| Silicified Microcrystalline | 211.9 | 190.69 |
| Cellulose | | |
| (HD90) | | |
| Croscarmellose Sodium | — | 37.5 |
| (Ac-Di-Sol) | | |
| Magnesium Stearate | 1.3 | 1.875 |
| Lactose | — | 75.0 |
| Layer Weight in mg | 260 | 375 |

— = not present

### Example 4

A bilayer tablet where the tetracycline (Meclocycline Sulfosalicylate) is in layer 1 and the buffer (sodium phosphate tribasic) is in layer 2.

| | Amount | Amount |
|---|---|---|
| | per | per |
| | Tablet | Tablet |
| | (mg) | (mg) |
| INGREDIENTS | Layer 1 | Layer 2 |
| Meclocycline Sulfosalicylate | 46.8 | — |
| Sodium Phosphate Tribasic | — | 70.0 |
| Silicified Microcrystalline | 159.9 | 190.69 |
| Cellulose | | |
| (HD90)) | | |
| Croscarmellose | 26.0 | 37.5 |
| (Ac-Di-Sol) | | |
| Magnesium Stearate | 1.3 | 1.875 |
| Lactose DT | 26.0 | 75.0 |
| Layer Weight in mg | 260 | 375 |

— = not present

### Example 5

A bilayer tablet where the tetracycline (Meclocycline Sulfosalicylate) is in layer 1 and the buffer (sodium phosphate tribasic) is in layer 2.

| | Amount | Amount |
|---|---|---|
| | per | per |
| | Tablet | Tablet |
| | (mg) | (mg) |
| INGREDIENTS | Layer 1 | Layer 2 |
| Meclocycline Sulfosalicylate | 46.8 | — |
| Sodium Phosphate Tribasic | — | 70.0 |
| Microcrystalline Cellulose PH200 | 251.3 | 228.2 |
| Croscarmellose Sodium | 37.5 | 37.5 |
| (Ac-Di-Sol) | | |
| Magnesium Stearate | 1.9 | 1.9 |
| Lactose High Velocity (DT) | 37.5 | 37.5 |
| Layer Weight in mg | 375 | 375 |

— = not present

### Example 6

A bilayer tablet where the tetracycline (Meclocycline Sulfosalicylate) is in layer 1 and the buffer (sodium phosphate tribasic) is in layer 2.

| | Amount | Amount |
|---|---|---|
| | per | per |
| | Tablet | Tablet |
| | (mg) | (mg) |
| INGREDIENTS | Layer 1 | Layer 2 |
| Meclocycline Sulfosalicylate | 46.8 | — |
| Sodium Phosphate Tribasic | — | 70.0 |
| Microcrystalline Cellulose PH200 | 251.3 | 168.5 |
| Croscarmellose Sodium | 37.5 | 30.0 |
| (Ac-Di-Sol) | | |
| Magnesium Stearate | 1.9 | 1.5 |
| Lactose | — | 75.0 |
| Lactose High Velocity (DT) | 37.5 | — |
| Sodium Starch Glycolate | — | 30.0 |
| (Explotab) | | |
| Layer Weight in mg | 375 | 300 |

— = not present

### Example 7

A bilayer tablet where the tetracycline (Meclocycline Sulfosalicylate) is in layer 1 and the buffer (sodium phosphate tribasic) is in layer 2.

| | Amount | Amount |
|---|---|---|
| | per | per |
| | Tablet | Tablet |
| | (mg) | (mg) |
| INGREDIENTS | Layer 1 | Layer 2 |
| Meclocycline Sulfosalicylate | 46.8 | — |
| Sodium Phosphate Tribasic | — | 70.0 |
| Microcrystalline Cellulose PH200 | 251.3 | 166.6 |
| Croscarmellose Sodium | 37.5 | 42.0 |
| (Ac-Di-Sol) | | |
| Magnesium Stearate | 1.9 | 1.4 |
| Lactose High Velocity (DT) | 37.5 | — |
| Sodium Starch Glycolate | — | 30.0 |
| (Explotab) | | |
| Layer Weight in mg | 375 | 280 |

— = not present

Examples 2-7 are of bilayer tablets. Of these examples, Example 2 has the best overall characteristics, i.e., the active blend has good flow, the tablet disintegrates quickly, the tablet is compressible, the friability is good, and high compression forces are not required to make the tablet. More specifically, Example 2 has better active blend flow and a faster disintegration time than Example 3. Example 2 has better active flow than Example 4. Difficulty was encountered preparing tablets of Example 5, due to the higher fill weight and the size of the die that was used, but no such difficulty was encountered preparing the tablets of Example 2. Example 2 was more compressible than Example 6, and Example 2 had a faster disintegration time. Example 2 did not need compression force as high as that in example 7, and Example 2 had better friability than Example 7.

It should be understood that the foregoing disclosure emphasizes certain specific embodiments of the invention and that all modifications or alternatives equivalent thereto are within the spirit and scope of the invention as set forth in the appended claims.

What is claimed is:

1. A pharmaceutical composition in the form of a multilayer tablet comprising
(a) a first region comprising Formulation (A), wherein Formula (A) comprises a therapeutically effective amount of a tetracycline, or a pharmaceutically acceptable salt thereof, in an amount of about 5% to about 40% by weight of Formulation (A), and a first carrier material comprising at least one pharmaceutically acceptable binder, carrier, adjuvant, excipient, diluent, disintegrant, or glidant; and
(b) a second region comprising Formulation (B), wherein Formulation (B) comprises a buffer and a second carrier material comprising at least one pharmaceutically acceptable binder, carrier, adjuvant, excipient, diluent, disintegrant, lubricant, or glidant, wherein the tablet rapidly disintegrates in an aqueous medium.

2. A pharmaceutical composition according to claim 1, wherein the buffer comprises a tribasic phosphate salt.

3. A pharmaceutical composition according to claim 1, wherein the second carrier material comprises microcrystalline cellulose.

4. A pharmaceutical composition according to claim 1, wherein the second carrier material comprises croscarmellose sodium.

5. A pharmaceutical composition according to claim 1, wherein the second carrier material comprises magnesium stearate.

6. A composition according to claim 1, wherein the tetracycline is meclocycline or a salt thereof.

7. A pharmaceutical composition according to claim 1, wherein the first carrier material comprises microcrystalline cellulose.

8. A pharmaceutical composition according to claim 1, wherein the first carrier material comprises croscarmellose sodium.

9. A pharmaceutical composition according to claim 1, wherein the first carrier material comprises magnesium stearate.

10. A composition according to claim 1, wherein the tetracycline is meclocycline sulfosalicylate.

11. A composition according to claim 1, wherein the tablet has a hardness of greater than 5 kp, friability less than 0.5% and disintegration time of less than 30 seconds.

12. A composition according to claim 1, wherein the first carrier material comprises microcrystalline cellulose, croscarmellose sodium, magnesium stearate, and lactose anhydrous high velocity.

13. A composition according to claim 1, wherein the second carrier material comprises microcrystalline cellulose, croscarmellose sodium, and magnesium stearate, wherein the microcrystalline cellulose is silicified microcrystalline cellulose.

14. A composition according to claim 1, in the form of a bilayer tablet, wherein
the first carrier material comprises about 150-350 mg of microcrystalline cellulose, about 20-60 mg of croscarmellose sodium, about 0.1-5 mg of magnesium stearate, and about 20-60 mg of lactose anhydrous high velocity; and
the second carrier material comprises about 100-400 mg of microcrystalline cellulose, about 20-60 mg of croscarmellose sodium, and about 0.1-5 mg of magnesium stearate, wherein the microcrystalline cellulose is silicified microcrystalline cellulose.

15. A composition according to claim 14, wherein the buffer is sodium phosphate tribasic.

16. A composition according to claim 14, wherein the weight of the first region is equal to or greater than the weight of the second region.

17. A composition according to claim 15, wherein
the first carrier material comprises about 200-300 mg of microcrystalline cellulose, about 30-45 mg of croscarmellose sodium, about 0.8-2.5 mg of magnesium stearate, and about 30-45 mg of lactose anhydrous high velocity;
the second carrier material comprises about 125-225 mg of microcrystalline cellulose, about 30-50 mg of croscarmellose sodium, and about 0.8-2.5 mg of magnesium stearate, wherein the microcrystalline cellulose is silicified microcrystalline cellulose;
the weight of the sodium phosphate tribasic is about 50-100 mg; and
the weight of the meclocycline is about 25-75 mg.

18. A composition according to claim 1, wherein at least one of the regions contains at least one disintegrant.

19. A method of treating oral mucositis (OM), comprising administering a pharmaceutical composition of claim 1 to a patient in need of such treatment.

20. A method according to claim 19, wherein the composition is dissolved in water and then administered to the oral cavity of the patient.

21. A method according to claim 20, wherein the water containing the dissolved composition has a pH of about 5.0-8.0.

22. A composition according to claim 1 in the form of a trilayer tablet, comprising
(a) a first region comprising Formulation (A), wherein Formulation (A) comprises a therapeutically effective amount of a tetracycline, or a pharmaceutically acceptable salt thereof, in an amount of about 5% to about 40% by weight of Formulation (A), and a first carrier material comprising at least one pharmaceutically acceptable binder, carrier, adjuvant, excipient, diluent, disintegrant, lubricant, or glidant;
(b) a second region comprising Formulation (B), wherein Formulation (B) comprises, a buffer and a second carrier material comprising at least one pharmaceutically acceptable binder, carrier, adjuvant, excipient, diluent, disintegrant, lubricant, or glidant, wherein the tablet rapidly disintegrates in an aqueous medium; and
(c) a third region, located between the first and second regions, where the third region comprises at least one pharmaceutically acceptable binder, carrier, adjuvant, excipient, diluent, disintegrant, lubricant, or glidant.

23. A composition according to claim 22, where the third region is a barrier layer isolating the first region from the second region.

24. A composition according to claim 23, where the tetracycline is meclocycline sulfosalicylate.

25. A composition according to claim 24, wherein the buffer is a tribasic phosphate salt or tromethamine.

26. A solution formed by adding a composition according to claim 1 to water.

27. A method for preparing a mouth rinse comprising adding a composition of claim 1 to water.

28. A method for treating or preventing mucositis comprising contacting a composition of claim 1 with an aqueous medium for a time sufficient to form a solution, contacting the solution to the oral cavity of a patient, and removing the solution from the patient's oral cavity.

29. A method according to claim 28, wherein the tetracycline is meclocycline.

30. A method according to claim 28, wherein the tetracycline is meclocycline sulfosalicylate and the buffer is a tribasic phosphate salt.

31. An aqueous formulation comprising (a) a solution phase comprising a solution comprising tetracycline and a buffer, and (b) a solid phase present or suspended in the solution phase, the solid phase comprising water insoluble material.

32. An aqueous formulation according to claim 31, where the water insoluble material comprises tablet binder, carrier, adjuvant, excipient, diluent, disintegrant, glidant, lubricant or combinations thereof.

33. A formulation of claim 1 that disintegrates within about 8 to 12 seconds of being added to an aqueous medium, and where about 90% of the tetracycline and buffer dissolves within about 30 seconds, when the aqueous medium is mixed.

34. An aqueous formulation having a volume of from about 5-25 ml and comprising (a) a solution phase comprising water, about 0.07-2% (w/w) of a tetracycline or salt thereof and about 0.1-4% (w/w) of a buffer, and (b) a solid phase present or suspended in the solution phase, the solid phase comprising water insoluble material.

35. A formulation according to claim 34, where the volume of the formulation is from about 10-20 ml.

36. A formulation according to claim 35, where the weight percentage of tetracycline in the solution phase is from about 0.1 to about 1.0.

37. A formulation according to claim 35, where the weight percentage of tetracycline in the solution phase is from about 0.15 to about 0.5.

38. A formulation according to claim 35, where the water insoluble material is tablet binder, carrier, adjuvant, excipient, diluent, disintegrant, glidant, lubricant or a combination thereof.
